(19) United States
(12) Patent Application Publication
(10) Pub. No.: US 2025/0295710 A1
(43) Pub. Date: Sep. 25, 2025

(54) PREBIOTIC AND PROBIOTIC FORMULATION CONTAINING ACID AND HEAT RESISTANT BACTERIA

(71) Applicants: Kedar Prasad, San Rafael, CA (US); Tyler Delarosa, New York, NY (US)

(72) Inventors: Kedar Prasad, San Rafael, CA (US); Tyler Delarosa, New York, NY (US)

(21) Appl. No.: 18/831,490

(22) Filed: Feb. 25, 2025

Related U.S. Application Data
(63) Continuation-in-part of application No. 18/445,613, filed on Nov. 21, 2023, now abandoned.

(51) Int. Cl. A61K 35/745; A61K 31/716; A61K 31/732; A61K 31/733; A61K 31/736; A61K 35/742; A61K 35/747; A61K 36/064; A61K 36/73; A61K 36/899

(52) U.S. Cl. CPC A61K 35/745; A61K 31/716; A61K 31/732; A61K 31/733; A61K 31/736; A61K 35/742; A61K 35/747; A61K 36/064; A61K 36/73; A61K 36/899

(57) ABSTRACT

A probiotic formulation comprises acid and heat resistant probiotics, like Saccharomyces boulardii and Bacillus coagulans.

### RELATED APPLICATIONS

This application is a continuation-in-part of U.S. application Ser. No. 18/445,613 filed on Nov. 21, 2023 and entitled “Probiotic Formulation for Intestinal Dysbiosis”.

### BACKGROUND

Even though Dr. Hippocrates, a physician, and a philosopher stated, “All diseases begin in the gut” some 2500 years ago, scientists became interested in investigating intestinal microorganisms and their role in human health and disease much later. It was not until the year 1880, when Dr. Theodor Escherich, an Austrian pediatrician, observed a bacterium (Escherich coli) in the intestine of healthy children and children who were suffering from diarrhea. Since then, identification and importance of microorganisms in the gut and other parts of the body continued. During the last two decades, several harmful and beneficial microorganisms were identified and their role in aging and age-related diseases including diabetes type II were studied. The current studies suggest that the intestines of normal individuals are colonized by a wide range of microbiota which include probiotics bacteria, viruses, fungi, and other micro-organisms. Beneficial probiotics bacteria include most species of Bifidobacterial and Lactobacillus, whereas harmful bacteria include some species of Colostridium, Enterococcus, Staphylococcus, Bacteroides, E. coli, Helicobacter pylori. The intestinal dysbiosis is defined by an imbalance in bacterial composition in which loss of beneficial bacteria, and overgrowth of potentially pathogenic bacteria occurs. The growth of harmful bacteria generates pro-inflammatory cytokines which are toxic to the cells, and deprive the body of certain B-vitamins, vitamin K, neurotransmitters, and short-chain fatty acids which are essential for maintaining good health. Intestinal dysbiosis is associated with Type 1 and Type 2 Diabetes. Intestinal dysbiosis also plays an important role in rapid progression of insulin resistance in diabetes type II. In addition, intestinal dysbiosis can decrease production of short-chain fatty acids such as butyric acid, propionic acid, and acetic acid due to decline in the number of butyric acid-producing bacteria in patients with diabetes type II. Butyric acid has diverse biological functions which include improving intestinal barrier integrity, pancreatic beta-cell proliferation, and insulin sensitivity, and reducing glycemia and body weight. Intestinal dysbiosis can also decrease production of other metabolites such as branched fatty acids which causes insulin resistance that can lead to the development of diabetes type II. Intestinal dysbiosis also causes inflammation and enhance intestinal permeability. Intestinal dysbiosis has been observed in animal models and in patients with diabetes type II and its complications such as retinopathy (eye damage), nephropathy (kidney damage), neuropathy (damage to peripheral Nerve), cardiovascular diseases, coronary artery disease (8). Severity of the gut dysbiosis is related to the severity of diabetes type II.

Therefore, restoring the composition of probiotics in favor of beneficial bacteria by probiotics with prebiotics would be needed to reduce the risk of diabetes type II and decrease the rate of progression of diabetes type I and type II. Administration of probiotics with prebiotics may reverse intestinal dysbiosis by increasing the number of beneficial bacteria and reducing the number of toxic bacteria, and thereby, reduce the risk of development and progression of diabetes. It is essential to add prebiotics with probiotics because they provide substrate to bacteria for fermentation which is necessary to produce short-chain fatty acids such as butyric acid.

U.S. Pat. No. 6,241,983 (“Paul et al”) relates to probiotics which are very sensitive to the acidic environment of the stomach and bile acid of the small intestine. Oral consumption of Paul's probiotics can lead to destruction of most probiotics while passing through the stomach and small intestine to the lower colon where they form colonies and grow. Therefore, there is little to no effectiveness of Paul's formula in reversing the effects of intestinal dysbiosis (increased population of harmful bacteria and decline in the number of beneficial bacteria) and in the treatment of intestinal inflammatory bowel syndrome. Unlike Paul and in order to improve the effectiveness of probiotics, the present invention utilizes acid and heat resistant probiotics, like Saccharomyces boulardii and Bacillus coagulans.

### SUMMARY OF THE INVENTION

In one embodiment, the present invention relates to a formulation comprising at least one beneficial bacteria and at least one prebiotic substrate wherein upon consumption of the formulation by a user, the beneficial bacteria ferments the prebiotic substrate.

In another embodiment, the beneficial bacteria is selected from the group consisting of Bacillus coagulans, yeast-derived Saccharomyces boulardii, Lactobacillus strain, Bifedobacterial strains, Lactobacillus plantarum, Lactobacillus rhamnosus, Lactobacillus salivarius, Lactobacillus reuteri, Lactobacillus gasseri, Lactobacillus acidophilus, Lactobacillus casei, Lactobacillus paracasei, Bifedobacterial bifidum, Bifedobacterial animalis, Bifedobacterial longum, Bifedobacterial breve, Bifedobacterium infantis, bacillus coagulans, and combinations and mixtures thereof.

In yet another embodiment, prebiotic substrate is selected from the group consisting of apple pectin, fiber, guar gum, inulin, oat beta glucan, larch arabingalactan, rice flour, nonstarch polysaccharides, galacto-oligosaccharides, and transgalacto-oligosacchrides, combination and mixtures thereof.

In still another embodiment, the number of said at least one beneficial bacteria in said formulation is from about 10 to about 25 per dose. In still yet another embodiment, the number of said at least one prebiotic agent in said formulation is from about 4 to about 12 per dose. In a further embodiment, a therapeutically effective amount of said formulation is intended to be administered as a probiotic.

In yet a further embodiment, the formulation further comprises a dispersant for creating smaller particles, said dispersant is selected from the group consisting of magnesium stearate, rice flour, organic dispersants, inorganic dispersants, and combinations and mixtures thereof.

In still a further embodiment, a therapeutically effective amount of the composition is intended to be administered once a day. In still yet a further embodiment, a therapeutically effective amount of the composition is intended to be administered twice a day. In another further embodiment, a therapeutically effective amount of the formulation is intended to be administered to a patient with intestinal dysbiosis.

In still another further embodiment, the Lactobacillus strain is selected from the group consisting of Lactobacillus rhamnosus, Lactobacillus salivarius, Lactobacillus acidophilus, Lactobacillus casei, Lactobacillus plantarum, Lactobacillus paracasei, Lactobacillus reuteri, Lactobacillus rhamnosus, Lactobacillus gasseri, and combinations and mixtures thereof. In yet another further embodiment, the Bifedobacterial strains is selected from the group consisting of Bifedobacterium longum, Bifedobacterium breve, Bifedobacterium bifidum, Bifedobacterium infantis, Bifedobacterial animalis, and combinations and mixtures thereof.

In another embodiment, the beneficial bacteria and the prebiotic agents are the only therapeutically active agents in the formulation.

In a further embodiment, the present invention relates to a formulation comprising at least one probiotic and at least one prebiotic, and the probiotic comprises at least one beneficial bacteria, and the prebiotic comprises at least one fermentation substrate. In another further embodiment, the beneficial bacteria is selected from the group consisting of Bacillus coagulans, Saccharomyces boulardii, Lactobacillus strain, Bifedobacterial strains, Lactobacillus plantarum, Lactobacillus rhamnosus, Lactobacillus salivarius, Lactobacillus reuteri, Lactobacillus gasseri, Lactobacillus acidophilus, Lactobacillus casei, Lactobacillus paracasei, Bifedobacterial bifidum, Bifedobacterial animalis, Bifedobacterial longum, Bifedobacterial breve, bacillus coagulans, and combinations and mixtures thereof.

In still another further embodiment, the Lactobacillus strain is selected from the group consisting of Lactobacillus rhamnosus, Lactobacillus salivarius, Lactobacillus acidophilus, Lactobacillus casei, Lactobacillus plantarum, Lactobacillus paracasei, Lactobacillus reuteri, Lactobacillus rhamnosus, Lactobacillus gasseri, and combinations and mixtures thereof.

In yet another further embodiment, the Bifedobacterial strains is selected from a group consisting of Bifedobacterium longum, Bifedobacterium breve, Bifedobacterium bifidum, Bifedobacterium infantis, Bifedobacterial animalis, and combinations and mixtures thereof.

In a further embodiment, the one fermentation substrate is selected from the group consisting of apple pectin, fiber, guar gum, inulin, oat beta glucan, larch arabingalactan, rice flour, apple juice, grape juice and orange juice, MRS agar, non-fermented milks, kimmed milk, soy milk and modified MRS broth, carbohydrates, and combination and mixtures thereof.

In another embodiment, the formulation further comprises a dispersant for creating smaller particles, said dispersant is selected from the group consisting of magnesium stearate, rice flour, organic dispersants, inorganic dispersants, and combinations and mixtures thereof. In another further embodiment, the formulation wherein upon consumption of the formulation by a user, the probiotic ferments the prebiotic.

In another embodiment, the present invention relates to a prebiotic and probiotic system consisting of: a prebiotic formulation consisting of Guar Gum, Inulin, Oat Beta Glucan, Larch Arabingalactan, Rice Flour, Apple Pectin and Fiber; and a probiotic formulation consisting of Bifidobacterium longum, Lactobacillus rhamnosus, Lactobacillus salivarius, Lactobacillus acidophilus, Lactobacillus casei, Lactobacillus plantarum, Lactobacillus paracasei, Bifidobacterium breve, Bifidobacterium lactis, Bifidobacterium bifidum, Bifidobacterium infantis, Lactobacillus reuteri, Bacillus coagulans,and Saccharomyces Boulardii.

In a further embodiment, the Bifedo and lactobacillus strains of bacteria are acid sensitive and from about 60 to about 70% of said Bifedo and lactobacillus strains of bacteria, when consumed, are killed by the acid pH of the stomach and the bile acid of the intestines.

In another further embodiment, the Saccharomyces boulardii and Bacillus coagulans are both acid and heat resistant and when consumed, the Saccharomyces boulardii and Bacillus coagulans survive the acid pH of the stomach and the bile acid of the intestines.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

The word Probiotic comes from the Greek meaning “for life”. The gut has a complex ecosystem in which nutrients, microbiota (refers to bacteria, viruses, and fungi), and host intestinal cells interact to improve human health. The probiotics are defined as microorganisms, which when administered in sufficient amounts provide health benefits to the host. Probiotics are live microorganisms that provide health benefits when consumed, generally by improving or restoring the gut microbiota. These live microorganisms are a combination of live beneficial bacteria and/or yeasts that naturally live in your body. Although viewed negatively, there are actually two forms of bacteria: good and bad bacteria. Probiotics are good bacteria that fight or balance the bad bacteria. When you are sick, bad bacteria has entered your body and multiplies in number, thereby knocking the body out of balance. Good bacteria or probiotics fights off the bad bacteria and restores balance within the body, making the individual feel better. In order to be a probiotic, it must include the following characteristics: be isolated from a human; survive in the intestine after digestion; have proven benefits; and can be consumed safely. The probiotics provide a plurality of health benefits: 1) probiotics help balance the friendly bacteria in the. digestive system; 2) probiotics can help prevent and treat diarrhea; 3) probiotics may help improve mental health conditions; 4) certain probiotic strains can help with heart health; 5) probiotics may reduce the severity of certain allergies and eczema; 6) probiotics may help reduce symptoms of certain digestive disorders; 7) probiotic may help boost the immune system; and 8) probiotics may help you lose weight and belly fat.

For purposes of this invention, prebiotic is any substrate that is selectively utilized by host microorganisms conferring a health benefit. Often, prebiotics are types of soluble fibers that humans cannot digest but instead serve as “food” for beneficial microbes that already live in the colon or elsewhere in the body. Fructo-oligosacchrides (FOS), galacto-oligosaccharides (GOS), and transgalacto-oligosacchrides (TOS) are common prebiotics. Fermentation of prebiotics by gut microbiota produces short-chain fatty acids, including lactic acid, butyric acid and propionic acid.

Bifedo or lactobacillus strains of bacteria which are acid sensitive and most of them (60-70%), when consumed, are killed by the acid pH of the stomach and the bile acid of the intestines. So for the purposes of this invention, Saccharomyces boulardii and Bacillus coagulans play significant roles because they survive 100%. Saccharomyces boulardii and Bacillus coagulans are both acid and heat resistant.

Probiotics Boost with Prebiotics has Following Beneficial Bacteria
1. Saccharomyces boulardii: This probiotic is a form of yeast derived from the fruit lychee. The fermented foods such as sauerkraut and kimchi are rich in Saccharomyces boulardii. It is superior to Lactobacillus and Bifidobacterium, because it is resistant to acid and temperature. The survival of this probiotic after gastric acidity is 75%. This probiotic grows optimally at 370C. The stomach pH is around 2. Lactobacillus and Bifedobacterial strains are very sensitive to this acidic pH. S. boulardii prevents infection from harmful microorganisms, improve gut barrier function, symptoms of inflammatory bowel diseases, Crohn's disease, ulcerative colitis, immune function, and increase production of short chain fatty acid such as butyric acid during fermentation of insoluble and soluble fibers. This probiotic does not enter the bloodstream and is removed from the gut in 5 days.
2. Lactobacillus and Bifidobacterial: Lactobacillus strains and bifidobacterial strains decreased the risk of colorectal cancer, improve the symptoms of inflammatory bowel disease, and alcoholic and non-alcoholic fatty liver diseases, reduce the risk of obesity, type II diabetes, oxidative stress-related chronic diseases, and immune-mediated diseases. Combination of two probiotics decreased acute stress and depression. Lactobacillus inhibited pro-inflammatory cytokines and attachment of harmful bacteria. Bifidobacterial strains improved the symptoms of irritable bowel disorders, increased production of short-chain fatty acids, such as butyric during fermentation of insoluble and soluble fibers, which participated in reducing the risk of chronic diseases, and improved memory function.
3. Bacillus Coagulans: This probiotic is found in the intestine. These probiotics are resistant to acid and temperature. Bacillus coagulans improves digestion, inhibits the growth of pathogenic bacteria, promotes the ability to form spores which can survive in harsh environment of the gut, helps in forming B-vitamins, improves immune system, attenuates inflammatory bowel disease (IBD) and irritable bowel syndrome (IBS), produces short-chain fatty acids such as butyric acid, and reduces the risk of metabolic diseases such as obesity and type II diabetes.

### Fermentation Product of Probiotics and Prebiotics

Butyric acid: Butyric acid is one of the short chain fatty acids, which is produced during fermentation of fibers by probiotics. Sodium butyrate inhibits the growth of cancer cells and enhances the effects of x-irradiation and chemotherapeutic agents. Importance of sodium butyrate was reviewed in 1980. Since then its beneficial effects were demonstrated in inflammatory bowel disorders by reducing pro-inflammatory cytokines, attenuates the risk of certain neurodegenerative diseases, diabetes, and cardiovascular diseases.

In one of the embodiments of the present invention, Table 1, set forth below, represents the ingredients, units, potency and dose for the Prebiotic formulation.

TABLE 1
Prebiotic Formulation:
Item Description:

| Active | Units | Potency | Dose |
|---|---|---|---|
| Guar Gum | 100 mg | 1.00 | 100.00 |
| Inulin | 150 mg | 1.00 | 150.00 |
| Oat Beta Glucan | 100 mg | 1.00 | 100.00 |
| Larch Arabingalactan | 100 mg | 1.00 | 100.00 |
| Rice Flour (Heat Treated) | 75 mg | | |

In another embodiment, Table 2 represents the ingredients, units, potency and dose for the Prebiotic and Probiotic formulation.

TABLE 2
Prebiotic and Probiotic Formulation
Item Description:

| Active | Units | | Potency | Dose |
|---|---|---|---|---|
| Bifidobacterium longum (100B CFU/gm) | 2.65 | Billion | 0.10 | 26.50 |
| Lactobacillus rhamnosus (200B CFU/gm) | 2.6 | Billion | 0.20 | 13.00 |
| Lactobacillus salivarius (300B CFU/gm) | 2.6 | Billion | 0.30 | 8.67 |
| Lactobacillus acidophilus La-14 (200B CFU/gm) | 2.5 | Billion | 0.20 | 12.50 |
| Lactobacillus casei Lc-11 (300B CFU/gm) | 2.5 | Billion | 0.30 | 8.33 |
| Lactobacillus plantarum (400B CFU/gm) | 1.78 | Billion | 0.40 | 4.45 |
| Lactobacillus paracasei (400B CFU/gm) | 1.78 | Billion | 0.40 | 4.45 |
| Bifidobacterium breve (300B CFU/gm) | 1.3 | Billion | 0.30 | 4.33 |
| Bifidobacterium lactis (300B CFU/gm) | 1.3 | Billion | 0.30 | 4.33 |
| Bifidobacterium bifidum Bb-06 (100B CFU/gm) | 1.29 | Billion | 0.10 | 12.90 |
| Bifidobacterium infantis (50B CFU/gm) | 0.89 | Billion | 0.05 | 17.80 |
| Lactobacillus reuteri (100B CFU/gm) | 0.42 | Billion | 0.10 | 4.20 |
| Bacillus coagulans (NLT 100 billion spores/g) | 0.42 | Billion | 0.10 | 4.20 |
| Saccharomyces Boulardii (20 B CFU/gm) | 0.085 | Billion | 0.02 | 4.25 |
| Apple Pectin and Fiber | 50 | mg | 1.00 | 50.00 |
| Guar Gum | 100 | mg | 1.00 | 100.00 |
| Inulin | 150 | mg | 1.00 | 150.00 |
| Oat Beta Glucan | 100 | mg | 1.00 | 100.00 |
| Larch Arabingalactan | 100 | mg | | |

In addition, the claimed invention is not directed to a product of nature. The combination of ingredients (dispersant, probiotic and prebiotic substrate) in the presently claimed invention is neither a product of nature or naturally occurring. Ingredients were specifically selected for particular performances, formulated to be a commercial product and the final formulation is admixed and encapsulated by a factory. The formulation of the presently claimed invention has characteristics or properties that are different from the naturally-occurring counterpart (in this case, the individual ingredients). For example, the formulation of the present invention “wherein upon consumption of said formulation by a user, said at least one beneficial probiotic ferments said at least one prebiotic substrate.” The effectiveness of this formulation is in reversing the effects of intestinal dysbiosis (increased population of harmful bacteria and decline in the number of beneficial bacteria) and in the treatment and reduction of intestinal inflammatory bowel syndrome. The Bifedo and lactobacillus strains of bacteria are acid sensitive and from about 60 to about 70% of said Bifedo and lactobacillus strains of bacteria, when consumed, are killed by the acid pH of the stomach and the bile acid of the intestines, while the Saccharomyces boulardii and Bacillus coagulans are both acid and heat resistant and when consumed, the Saccharomyces boulardii and Bacillus Coagulans survive the acid pH of the stomach and the bile acid of the intestines.

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various objects and features thereof.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. A prebiotic and probiotic system consisting of:
a prebiotic formulation consisting of Guar Gum, Inulin, Oat Beta Glucan, Larch Arabingalactan, Rice Flour, Apple Pectin and Fiber; and
a probiotic formulation consisting of Bifidobacterium longum, Lactobacillus rhamnosus, Lactobacillus salivarius, Lactobacillus acidophilus, Lactobacillus casei, Lactobacillus plantarum, Lactobacillus paracasei, Bifidobacterium breve, Bifidobacterium lactis, Bifidobacterium bifidum, Bifidobacterium infantis, Lactobacillus reuteri, Bacillus coagulans, and Saccharomyces Boulardii.

2. The system of claim 1 wherein said Bifedo and lactobacillus strains of bacteria are acid sensitive and from about 60 to about 70% of said Bifedo and lactobacillus strains of bacteria, when consumed, are killed by the acid pH of the stomach and the bile acid of the intestines.

3. The system of claim 2 wherein said Saccharomyces boulardii and said Bacillus coagulans are both acid and heat resistant and when consumed, said Saccharomyces boulardii and said Bacillus coagulans survive the acid pH of the stomach and the bile acid of the intestines.

4. A formulation consisting of at least one dispersant, at least one beneficial probiotic and at least one prebiotic substrate wherein upon consumption of said formulation by a user, said at least one beneficial probiotic ferments said at least one prebiotic substrate, said at least one dispersant is used for creating small particles, said at least one beneficial probiotic is selected from the group consisting of Bacillus coagulans, Saccharomyces boulardii, Lactobacillus strain, Bifedobacterial strains, Lactobacillus plantarum, Lactobacillus rhamnosus, Lactobacillus salivarius, Lactobacillus reuteri, Lactobacillus gasseri, Lactobacillus acidophilus, Lactobacillus casei, Lactobacillus paracasei, Bifedobacterial bifidum, Bifedobacterial animalis, Bifedobacterial longum, Bifedobacterial breve, Bifedobacterium infantis, and combinations and mixtures thereof, said at least one prebiotic substrate is selected from the group consisting of apple pectin, guar gum, inulin, oat beta glucan, larch arabingalactan, rice flour, nonstarch polysaccharides, galacto-oligo saccharides, and transgalacto-oligosacchrides, combination and mixtures thereof, said dispersant is selected from the group consisting of magnesium stearate, rice flour, organic dispersants, inorganic dispersants, and combinations and mixtures thereof, wherein said Bifedo and lactobacillus strains of bacteria are acid sensitive and from about 60 to about 70% of said Bifedo and lactobacillus strains of bacteria, when consumed, are killed by the acid pH of the stomach and the bile acid of the intestines, and said Saccharomyces boulardii and said Bacillus coagulans are both acid and heat resistant and when consumed, said Saccharomyces boulardii and said Bacillus coagulans survive the acid pH of the stomach and the bile acid of the intestines.

5. The formulation of claim 4 wherein the number of said at least one beneficial probiotic in said formulation is from about 10 CFU to about 25 CFU per dose.

6. The formulation of claim 4 wherein the number of said at least one prebiotic substrate in said formulation is from about 4 CFU to about 12 CFU per dose.

7. The formulation of claim 4 wherein a therapeutically effective amount of said formulation is intended to be administered once a day.

9. The formulation of claim 4 wherein a therapeutically effective amount of said formulation is intended to be administered twice a day.

10. The formulation of claim 1 wherein a therapeutically effective amount of said formulation is intended to be administered to a patient with intestinal dysbiosis.

11. The formulation of claim 4 wherein said Lactobacillus strain is selected from the group consisting of Lactobacillus rhamnosus, Lactobacillus salivarius, Lactobacillus acidophilus, Lactobacillus casei, Lactobacillus plantarum, Lactobacillus paracasei, Lactobacillus reuteri, Lactobacillus gasseri, and combinations and mixtures thereof.

12. The formulation of claim 4 wherein said Bifedobacterial strains is selected from the group consisting of Bifedobacterium longum, Bifedobacterium breve, Bifedobacterium bifidum, Bifedobacterium infantis, Bifedobacterial animalis, and combinations and mixtures thereof.

13. A probiotic formulation consisting of Bifidobacterium longum, Lactobacillus rhamnosus, Lactobacillus salivarius, Lactobacillus acidophilus, Lactobacillus casei, Lactobacillus plantarum, Lactobacillus paracasei, Bifidobacterium breve, Bifidobacterium lactis, Bifidobacterium bifidum, Bifidobacterium infantis, Lactobacillus reuteri, Bacillus coagulans, and Saccharomyces Boulardii.

14. The formulation of claim 13 wherein said Bifedo and lactobacillus strains of bacteria are acid sensitive and from about 60 to about 70% of said Bifedo and lactobacillus strains of bacteria, when consumed, are killed by the acid pH of the stomach and the bile acid of the intestines.

15. The formulation of claim 14 wherein said Saccharomyces boulardii and said Bacillus coagulans are both acid and heat resistant and when consumed, said Saccharomyces boulardii and said Bacillus coagulans survive the acid pH of the stomach and the bile acid of the intestines.

16. The formulation of claim 13 wherein a therapeutically effective amount of said formulation is intended to be administered once a day.

17. The formulation of claim 13 wherein a therapeutically effective amount of said formulation is intended to be administered twice a day.

18. The formulation of claim 13 wherein a therapeutically effective amount of said formulation is intended to be administered to a patient with intestinal dysbiosis.
